(19) United States
(12) Patent Application Publication
(10) Pub. No.: US 2009/0304640 A1
(43) Pub. Date: Dec. 10, 2009

(54) Transfection of Collagen-Producing Cells

(75) Inventors: Ashish D. Diwan, Sydney (AU); Sylvia A. Chung, Arncliff (AU)

Correspondence Address: HOWSON & HOWSON LLP, 501 OFFICE CENTER DRIVE, SUITE 210, FORT WASHINGTON, PA, 19034, US

(73) Assignee: CELLIXE PTY LTD, Rockdale (AU)

(21) Appl. No.: 11/919,406

(22) PCT Filed: Apr. 27, 2006
(86) PCT No.: PCT/AU2006/000550

§ 371 Date: Apr. 9, 2008

Related U.S. Application Data
(60) Provisional application No. 60/675,814, filed on Apr. 27, 2005.

(51) Int. Cl. A61K 35/12; C12N 5/02; C12N 5/10; C12P 21/06; A61K 31/7088

(52) U.S. Cl. 424/93.7; 435/375; 435/325; 435/69.1; 514/44 R

(57) ABSTRACT

A method is provided for introducing at least one polynucleotide of interest into an intervertebral disc cell, wherein the polynucleotide of interest is transfected with a lipid-based delivery agent. Also provided are methods for introducing polynucleotides encoding one or more telomerase catalytic subunits into collagen-producing cells and methods for increasing extracellular matrix formation in collagen-producing cells.

### FIELD OF THE INVENTION

The present invention relates to methods for transfecting collagen-producing cells, in particular intervertebral disc cells. The present invention also relates to methods for the transfection of collagen-producing cells with polynucleotides encoding telomerase catalytic subunits and to methods and compositions for increasing extracellular matrix formation or lifespan of collagen-producing cells and to treating or preventing conditions associated with impaired activity of such cells.

### BACKGROUND OF THE INVENTION

The extracellular matrix in vertebrates is a complex mixture of primarily proteins and proteoglycans. Collagen is the predominant protein of the extracellular matrix and is an essential component of all connective tissue, including cartilage, bone, skin, ligaments and tendons. Collagens constitute a family of fibrous proteins imparting structural support and tensile strength to connective tissue, and are produced by a variety of connective tissue cells including principally fibroblasts, chondroblasts and chondrocytes.

Within the vertebral column collagen plays a vital role in the intervertebral discs. The intervertebral disc comprises two main regions, a central nucleus pulposus surrounded by an outer annulus fibrosis and is composed of chrondrocyte- and fibroblast-like cells embedded within a matrix comprised largely of highly organised collagen fibres (see for example Chung, S. A. et al. 2003, The molecular basis of intervertebral disc degeneration, Orthop Clin N Am 34:209-219). Stability and strength provided by the extracellular matrix in the intervertebral disc is largely dependent on the structure and content of collagen fibres and the interaction of these fibres with other matrix components. Degeneration of the intervertebral disc is due largely to changes to the structure and content of collagen fibres in the extracellular matrix. For example during degeneration collagen fibres become damaged resulting in disruption of the normally highly oriented organization of fibrils (reviewed in Chung, S. A. et al. 2003, The molecular basis of intervertebral disc degeneration, Orthop Clin N Am 34:209-219). Cells within the intervertebral disc responsible for its maintenance and health deteriorate with age, losing synthetic capacity for essential components such as proteoglycan, aggrecan and collagen. Synchronised with the loss of extracellular matrix is dehydration of the nucleus pulposus, with severe consequences for the biology and biomechanical stability of the disc.

Intervertebral disc degeneration is implicated as a causative factor in a number of spinal disorder such as spinal stenosis, spinal segmental instability and disc herniations. Degeneration is also widely believed to be a significant factor leading to lower back pain. Chronic lower back pain is a major healthcare problem, estimated to afflict more than 500% of the population in the United States the United States alone and costing healthcare systems billions of dollars annually (Diwan, A. D. et al. 2000, Current concepts in intervertebral disk restoration, Orthop Clin N Am 31:453-464).

Accordingly, there is a need for effective treatments and preventative measures for intervertebral disc degeneration and disc-related spinal disorders.

Existing and presently proposed therapies for the repair or regeneration of the intervertebral disc focus on replacement, for example in the form of artificial intervertebral disc devices or prosthetic nucleus pulposus or regeneration of intervertebral disc by tissue engineering (reviewed in Diwan, A. D. et al. 2000, Current concepts in intervertebral disk restoration, Orthop Clin N Am 31:453-464). Whilst offering some promise, these therapies are still largely in their infancy. Additionally, in focusing on the alleviation of the direct symptoms of disc degeneration and associated disorders rather than on the treatment of the underlying causative factors, the potential remains for the recurrence of the problem following treatment.

There is a need for the development of alternative therapies targeting the molecular bases and morphological changes underlying intervertebral disc degeneration to offer prospects for improved long term treatments and prevention.

The present invention is predicated in part on the present inventors' discovery of a relationship between telomerase expression and collagen production in nucleus pulposus cells. In the course of their investigations the present inventors have also developed improved methods for the transfection of collagen-producing cells such as those of the intervertebral disc.

### SUMMARY OF THE INVENTION

According to a first aspect of the present invention there is provided a method for introducing at least one polynucleotide of interest into an intervertebral disc cell, wherein the polynucleotide of interest is transfected with a lipid-based delivery agent.

The intervertebral disc cell may be a cell of the nucleus pulposus or a cell of the annulus fibrosus. The intervertebral disc cell may be a fibroblast, chondroblast or chondrocyte.

The lipid based delivery agent may be a cationic lipid. The cationic lipid may be Lipofectamine 2000.

The lipid may be in the form of a liposome.

The polynucleotide of interest may be located within a vector and may be operably linked to a promoter active in the cell to be transfected.

The polynucleotide may encode one or more telomerase catalytic subunits. The telomerase catalytic subunit may be the telomerase reverse transcriptase encoded by the TERT gene. The telomerase reverse transcriptase may comprise the amino acid sequence as set forth in SEQ ID NO:1, The TERT gene may comprise the nucleotide sequence as set forth in SEQ ID NO:2.

According to a second aspect of the present invention there is provided a method for introducing at least one polynucleotide encoding one or more telomerase catalytic subunits into a collagen-producing cell, wherein the polynucleotide is transfected with a lipid-based delivery agent.

The collagen-producing cell may be an intervertebral disc cell. The intervertebral disc cell may be a cell of the nucleus pulposus or a cell of the annulus fibrosus. The cell may be a fibroblast, chondroblast or chondrocyte. The cell may be any pluripotent or totipotent cell capable of becoming a collagen-producing cell such as a mesenchymal stem cell, adult peripheral blood stem cell or embryonic stem cell.

The lipid based delivery agent may be a cationic lipid. The cationic lipid may be Lipofectamine 2000.

The lipid may be in the form of a liposome.

The polynucleotide encoding one or more telomerase catalytic subunits may be located within a vector and may be operably linked to a promoter active in the cell to be transfected.

The telomerase catalytic subunit may be the telomerase reverse transcriptase encoded by the TERT gene. The telomerase reverse transcriptase may comprise the amino acid sequence as set forth in SEQ ID NO:1. The TERT gene may comprise the nucleotide sequence as set forth in SEQ ID NO:2.

According to a third aspect of the present invention there is provided an isolated collagen-producing cell transfected with at least one polynucleotide encoding one or more telomerase catalytic subunits.

The collagen-producing cell may be an intervertebral disc cell.

The telomerase catalytic subunit may be the telomerase reverse transcriptase. The telomerase reverse transcriptase may be encoded by the human TERT gene.

The polynucleotide may be transfected in the presence of a lipid-based delivery agent. The lipid based delivery agent may be Lipofectamine 2000.

According to a fourth aspect of the present invention there is provided a method for increasing collagen expression in a collagen-producing cell, the method comprising introducing into the cell at least one polynucleotide encoding one or more telomerase catalytic subunits.

The collagen-producing cell may be an intervertebral disc cell.

According to a fifth aspect of the present invention there is provided a method for increasing collagen expression in a patient in need thereof, the method comprising introducing into one or more collagen-producing cells of the patient at least one polynucleotide encoding one or more telomerase catalytic subunits.

Typically the polynucleotide(s) is introduced into the cells ex vivo and the transfected cells re-introduced into the patient.

The collagen-producing cells may be intervertebral disc cells or pluripotent or totipotent cells such as mesenchymal stem cells, adult peripheral blood stem cells or embryonic stem cells.

According to a sixth aspect of the present invention there is provided a method for increasing collagen expression in a patient in need thereof, the method comprising introducing into the patient one or more collagen-producing cells transfected with at least one polynucleotide encoding one or more telomerase catalytic subunits.

Typically the polynucleotide(s) is introduced into the cells ex vivo. The cells may be autologous or allogeneic.

The collagen-producing cells may be intervertebral disc cells or pluripotent or totipotent cells such as mesenchymal stem cells, adult peripheral blood stem cells or embryonic stem cells.

According to a seventh aspect of the present invention there is provided a method for increasing extracellular matrix formation by a collagen-producing cell, the method comprising introducing into the cell at least one polynucleotide encoding one or more telomerase catalytic subunits.

The collagen-producing cell may be an intervertebral disc cell.

According to an eighth aspect of the present invention there is provided a method for increasing extracellular matrix formation in a patient in need thereof, the method comprising introducing into one or more collagen-producing cells of the patient at least one polynucleotide encoding one or more telomerase catalytic subunits.

Typically the polynucleotide(s) is introduced into the cells ex vivo and the transfected cells re-introduced into the patient.

The collagen-producing cells may be intervertebral disc cells or pluripotent or totipotent cells such as mesenchymal stem cells, adult peripheral blood stem cells or embryonic stem cells.

According to a ninth aspect of the present invention there is provided a method for increasing extracellular matrix formation in a patient in need thereof, the method comprising introducing into the patient one or more collagen-producing cells transfected with at least one polynucleotide encoding one or more telomerase catalytic subunits.

Typically the polynucleotide(s) is introduced into the cells ex vivo. The cells may be autologous or allogeneic.

The collagen-producing cells may be intervertebral disc cells or pluripotent or totipotent cells such as mesenchymal stem cells, adult peripheral blood stem cells or embryonic stem cells.

According to a tenth aspect of the present invention there is provided a method for increasing the lifespan of a collagen-producing cell, the method comprising introducing into the cell at least one polynucleotide encoding one or more telomerase catalytic subunits.

The collagen-producing cell may be an intervertebral disc cell.

According to an eleventh aspect of the present invention there is provided a method for treating or preventing a condition associated with impaired collagen-producing cell activity in an individual, the method comprising administering to the individual an effective amount of at least one polynucleotide encoding one or more telomerase catalytic subunits.

Typically cells are transfected with the polynucleotide(s) ex vivo and subsequently introduced into the individual. The cells may be autologous or allogeneic.

The cells may be intervertebral disc cells or pluripotent or totipotent cells such as mesenchymal stem cells, adult peripheral blood stem cells or embryonic stem cells.

According to a twelfth aspect of the present invention there is provided a method for treating or preventing intervertebral disc degeneration, or a condition or disease associated with intervertebral disc degeneration in an individual, the method comprising administering to the individual an effective amount of at least one polynucleotide encoding one or more telomerase catalytic subunits.

In one embodiment the polynucleotide(s) is transfected into cells ex vivo and the transfected cells introduced into the individual. The cells may be autologous or allogeneic. The cells may be intervertebral disc cells or pluripotent or totipotent cells such as mesenchymal stem cells, adult peripheral blood stem cells or embryonic stem cells.

The condition or disease associated with intervertebral disc degeneration may be, for example, discogenic axial pain including back and neck pain, foraminal stenosis, malalignment, spinal segmental instability, spinal stenosis, or a disc herniation, such as a herniation of the nucleus pulposus.

The polynucleotide may be transfected into the cellular subunit of a tissue engineered component to replace one or more parts of the intervertebral disc.

According to a thirteenth aspect of the present invention there is provided a method for treating or preventing axial pain associated with intervertebral disc degeneration in an individual, the method comprising administering to the individual an effective amount of at least one polynucleotide encoding one or more telomerase catalytic subunits.

The axial pain may be back and neck pain.

Typically the polynucleotide is transfected into cells ex vivo and the transfected cells introduced into the individual. The cells may be autologous or allogeneic. The cells may be intervertebral disc cells or pluripotent or totipotent cells such as mesenchymal stem cells, adult peripheral blood stem cells or embryonic stem cells.

According to a fourteenth aspect of the present invention there is provided a composition for treating or preventing a condition associated with impaired collagen-producing cell activity in an individual, the composition comprising at least one polynucleotide encoding one or more telomerase catalytic subunits, optionally together with a lipid-based delivery agent.

According to a fifteenth aspect of the present invention there is provided a composition for treating or preventing intervertebral disc degeneration, or a condition or disease associated with intervertebral disc degeneration in an individual, the composition comprising at least one polynucleotide encoding one or more telomerase catalytic subunits, optionally together with a lipid-based delivery agent.

According to a sixteenth aspect of the present invention there is provided a composition for treating or preventing axial pain including back and neck pain associated with intervertebral disc degeneration in an individual, the composition comprising at least one polynucleotide encoding one or more telomerase catalytic subunits, optionally together with a lipid-based delivery agent.

According to a seventeenth aspect of the present invention there is provided a kit comprising at least one polynucleotide encoding one or more telomerase catalytic subunits together with a lipid-based delivery agent for transfection of said polynucleotide(s).

### DEFINITIONS

In this specification, unless the context requires otherwise, the word “comprise”, and variations such as “comprises” and “comprising”, will be understood to imply the inclusion of a stated integer or step or group of integers or steps but not the exclusion of any other integer or step or group of integers or steps.

As used herein the term “polynucleotide” refers to a single- or double-stranded polymer of deoxyribonucleotide bases, ribonucleotide bases or known analogues of natural nucleotides, or mixtures thereof.

As used herein the term “intervertebral disc” refers to any of the fibrocartilaginous discs separating vertebral bodies of the spine, from the cervical, thoracic, lumbar and lumbosacral regions.

As used herein the terms “treating” and “treatment” refer to any and all uses which remedy a disorder or disease state or symptoms, prevent the establishment of a disorder or disease, or otherwise prevent, hinder, retard, or reverse the progression of a disorder or disease or other undesirable symptoms in any way whatsoever.

As used herein the term “effective amount” includes within its meaning a non-toxic but sufficient amount of an agent or compound to provide the desired therapeutic or preventative effect. The exact amount required will vary from subject to subject depending on factors such as the species being treated, the age and general condition of the subject, the severity of the condition being treated, the particular agent being administered and the mode of administration and so forth. Thus, it is not possible to specify an exact “effective amount”. However, for any given case, an appropriate “effective amount” may be determined by one of ordinary skill in the art using only routine experimentation.

### BRIEF DESCRIPTION OF THE DRAWINGS

The present invention will now be described with reference, by way of example only, to the following drawings.

FIG. 1. Transfection efficiency of lipofectamine-mediated delivery of DNA into sheep annulus fibrosus cells. 3-dimensional graph of β-galactosidase activity (milliunits) resulting from transfection of 80-320 ng pCMV.SPORT-βgal in 0.4-1 μl Lipofectamine 2000 reagent.

FIG. 2. Telomerase activity in sheep nucleus pulposus cells following transfection with hTERT. Relative telomerase activity (RTA %) 24 hours after transfection of cells with 240 ng hTERT-containing vector or vector only in 0.6 μl Lipofectamine 2000.

FIG. 3. Telomere length in sheep nucleus pulposus cells following transfection with hTERT. (A) Southern blot analysis of genomic DNA from cells 24 hours after transfection with 240 ng hTERT-containing vector or vector only in 0.6 μl Lipofectamine 2000. Lanes 1 and 6, molecular weight (kbp) markers (MW); lane 2, vector only transfected cells; lane 3, vector containing hTERT transfected cells; lane 4, short telomere control; Lane 5, long telomere control. (B) Southern blot analysis of genomic DNA from cells transfected with 240 ng hTERT-containing vector (hTERT) or vector only (control) in 0.6 μl Lipofectamine 2000, 73 days, 278 days and 329 days post-transfection.

FIG. 4. Population doubling of sheep nucleus pulposus cells transfected with hTERT. Cumulative population doubling of transfected cells between 70 and 520 days post transfection with 240 ng hTERT-containing vector or vector only in 0.6 μl Lipofectamine 2000.

FIG. 5. Matrix expression in sheep nucleus pulposus cells transfected with hTERT. Real-time PCR analysis of the relative expression of type-I collagen (COL1), type-II (COL2) collagen and aggrecan (AGG) in cells from days 87 to 424 post transfection with 240 ng hTERT-containing vector compared to vector only in 0.6 μl Lipofectamine 2000.

FIG. 6. Proliferation of sheep nucleus pulposus cells following transfection with hTERT. Metabolism of the tetrazolium salt MTS (absorbance at 490 nm) in cells transfected with 240 ng hTERT-containing vector or vector only in 0.6 μl Lipofectamine 2000 on day 397 (passage 24) post transfection.

FIG. 7. Senescence of sheep nucleus pulposus cells following transfection with hTERT. A and B, Illustrative micrographs of cells stained for β-galactosidase activity at pH 6 on day 439 (passage 26) post transfection with 240 ng hTERT-containing vector (A) or vector only (B) in 0.6 μl Lipofectamine 2000. C, Graphical representation of the percentage stained cells in hTERT-containing vector and vector only samples.

FIG. 8. In vitro transformation. A, Flow cytometry analysis of cell cycle arrest in hTERT-transfected nucleus pulposus cells, untransfected (parental) nucleus pulposus cells and HeLa cells in the presence (+) and absence (−) of actinomycin-D. Figures represent the percentage of is cells remaining in S-phase. B, Western blot analysis of p53 expression in hTERT-transfected nucleus pulposus cells, untransfected (parental) nucleus pulposus cells and HeLa cells in the presence (+) and absence (−) of actinomycin-D. C, Anchorage independent growth of agar suspension cultures of hTERT-transfected nucleus pulposus cells, untransfected (parental) nucleus pulposus cells and HeLa cells.

The amino acid sequence set forth in SEQ ID NO:1 is the amino acid sequence of human telomerase reverse transcriptase (GenBank Accession No. AF018167).

The nucleotide sequence set forth in SEQ ID NO:2 is the nucleotide sequence of the human telomerase reverse transcriptase gene (hTERT) (GenBank Accession No. AF018167).

The sequences of oligonucleotide primers used in the present studies are set forth in SEQ ID NOs:3 to 10.

### DETAILED DESCRIPTION OF EMBODIMENTS OF THE INVENTION

Telomeres exist as protein—DNA complexes physically located at, and defining, the ends of chromosomes. Telomeric DNA consists of multiple (typically thousands) tandem repeats of short sequences to which various proteins bind, thereby constituting the telomere complex. The length and integrity of the telomeres at the ends of chromosomes directly relate to the longevity of a cell containing those chromosomes. The shortening of telomeres is known to correlate with reduced cellular lifespan and the induction of cell senescence. Various methods for determining and measuring telomere length are known to those skilled in the art.

A specific ribonucleoprotein, telomerase, is responsible for maintaining and regulating telomere length. A portion of the telomerase RNA moiety is used as a template to direct synthesis of telomeric DNA repeat sequences by the catalytic protein component of the telomerase. This catalytic subunit is known as telomerase reverse transcriptase (TERT). The human TERT (hTERT) gene has been cloned and sequenced (see for example Cech et al., U.S. Pat. No. 6,166,178 and Cech et al., U.S. Pat. No. 6,617,110).

The present inventors have now surprisingly found, as disclosed herein, that the expression of type-I and type-II collagen is increased in intervertebral disc cells transfected with a vector containing the hTERT gene as compared to cells transfected with vector only. The expression of exogenous hTERT in the transfected cells also delayed cellular senescence thereby extending cellular population doubling. The cellular phenotype of the hTERT-transfected intervertebral disc cells was spindle-shaped, while the vector only-transfected cells possessed large cytoplasmic regions typical of senescenced cells.

The present inventors have for the first time established a relationship between telomerase expression and collagen expression in intervertebral disc cells, thus opening the way for a number of therapeutic possibilities for treating and/or preventing conditions associated with defects in the intervertebral disc. As exemplified herein, expression of telomerase in nucleus pulposus cells resulted in an extension of cellular lifespan allowing the prolonged maintenance of matrix production by nucleus pulposus cells, with expression of both collagen I and II being maintained from 142 to 424 days (a total of 282 days), compared to controls.

Methods of the present invention for transfecting cells with polynucleotides encoding one or more telomerase catalytic subunits are exemplified herein in relation to cells of the intervertebral disc. However those skilled in the art will readily appreciate that methods of the present invention find application in a variety of different collagen-producing cells, and cells capable of differentiating into collagen-producing cells, and thus can be used in treating and/or preventing defects of such cells.

Further, in carrying out these investigations the inventors have developed improved methods for the transfection of intervertebral disc cells with any polynucleotide of interest. In the experiments described herein transfection has been successfully achieved using a cationic lipid as the delivery vehicle for the polynucleotides.

The transfection of intervertebral disc cells has posed challenges in the past, possibly due to the rapid production and secretion of extracellular matrix material by these cells, interfering with the transfection process.

To date, transfection of the intervertebral disc has been achieved using adenoviral vector-based transfection methods. However adenoviral-mediated transfection suffers from a number of disadvantages making it often unsuitable for therapeutic applications, including the possibility of immunogenicity leading to the induction of a host immune response. Accordingly there is a need for alternative transfection methods, which do not require the use of viral or viral vector-based systems. However to date transfection of intervertebral disc cells has not been achieved using alternative methods.

The present inventors have now successfully achieved high efficiency transfection of intervertebral disc cells using a cationic lipid as the delivery vehicle for the polynucleotide(s) to be introduced. Using lipofectamine 2000 reagent as an example, optimal ratios of DNA:lipid for transfection have been established.

Accordingly, one aspect of the present invention relates to methods for the introduction of a polynucleotide(s) of interest into an intervertebral disc cell, wherein the polynucleotide of interest is transfected with a lipid-based delivery agent. The intervertebral disc cells may be any intervertebral disc cells, including but not limited to fibroblasts, chondroblasts, chondrocytes such as those of the nucleus pulposus or annulus fibrosus.

Another aspect of the invention relates to methods for introducing a polynucleotide encoding one or more telomerase catalytic subunits into collagen-producing cells, wherein the polynucleotide is transfected with a lipid-based delivery agent. The collagen-producing cells may be any collagen-producing cells, including but not limited to fibroblasts, chondroblasts, chondrocytes. The cells may be intervertebral disc cells such as those of the nucleus pulposus or annulus fibrosus. Alternatively, the cells may be pluripotent or totipotent cells such as, for example, mesenchymal stem cells, adult peripheral blood stem cells or embryonic stem cells.

Suitable methods are exemplified herein for the transfection of a reporter gene (encoding β-galactosidase) and the hTERT gene, although those skilled in the art will readily appreciate that the methods of the invention are applicable to the transfection of any polynucleotide of interest. The polynucleotide may be single-stranded or double-stranded and may, for example, encode a polypeptide or fragment thereof in which the target cell is deficient or may, for example, comprise an oligonucleotide, such as an antisense olignucleotide designed to inhibit expression of a target cellular nucleic acid. The person skilled in the art will appreciate that other polynucleotides are also suitable for use in the methods of the present invention.

Typically a polynucleotide to be transfected will be located in a vector. In such situations the polynucleotide is typically operably linked to one or more regulatory sequences, comprising for example a promoter sequence and/or at least one terminator sequence, such that the appropriate polypeptide sequence is produced following transfection of the polynucleotide. The vector may be a plasmid vector, or any other suitable vehicle adapted for the insertion of foreign sequences, their introduction into eukaryotic cells and the expression of the introduced sequences. Typically the vector is a eukaryotic expression vector and may include expression control and processing sequences such as a promoter, an enhancer, ribosome binding sites, polyadenylation signals and transcription termination sequences. The vector may also include one or more selectable markers, such as antibiotic resistance genes.

The vector may be episomal such that the polynucleotide of interest contained therein is maintained extra-chromosomally thereby eliminating any potential detrimental effects of chromosomal integration. Alternatively, the vector may enable or facilitate integration of the polynucleotide of interest into the genome. The choice of an episomal or integrating vector will depend largely on the particular circumstances, for example the cell type to be transfected and the polynucleotide of interest.

According to the methods of the present invention, polynucleotides are typically transfected in the presence of one or more lipid-based delivery agents, such as cationic lipids or phospholipids. Cationic lipids may be mono- or polycationic. Lipid-mediated DNA transfection is referred to as lipofection. Techniques and procedures for lipofection are known to those skilled in the art.

Cationic lipids suitable for use in methods of the invention include Lipofectamine 2000 (a commercial reagent comprising 2,3-dioleyloxy-N-[2(sperminecarboxyamido)ethyl]-N,N-dimethyl-1-propanaminium trifluoroacetate), Lipofectin, Lipofectace, DOTAP, DOTMA (N-[1-(2,3-dioleyloxy)propyl]-N,N,N-trimethylammonium chloride), CDAB (cetyldimethylethylammonium bromide), CTAB (cetyltrimethylethylammonium bromide), DDAB (dimethyldioctadecylammonium bromide), MBC (methylbenzethonium chloride), FuGENE (Roche) and stearylamine.

Other potentially suitable lipids are disclosed, for example in U.S. Pat. No. 5,855,910 (Ashley et al.), International Patent Application WO 02/072068 (Springer et al.) and International Patent Application WO 00/30444 (Xiang), the disclosures of which are incorporated herein by reference.

The lipid-based delivery agent may be formulated with the polynucleotide to be transfected as a liposome. Liposomes are generally derived from one or more phospholipids or other lipid substances, and are formed by mono- or multi-lamellar hydrated liquid crystals that are dispersed in an aqueous medium. Any non-toxic, physiologically acceptable and metabolisable lipid capable of forming liposomes can be used. The compositions in liposome form may contain stabilisers, preservatives, excipients and the like. Those of skill in the art will be able to determine suitable liposome formulations using routine procedures without undue experimentation. Methods for preparing liposomes are known in the art, and in relation to this specific reference is made to Gregoriadis, Ed., Liposome Technology, Volumes 1-3, CRC Press (1993) and Prescott, Ed., Methods in Cell Biology, Volume XIV, Academic Press, New York, N.Y. (1976), p. 33 et seq., the contents of which are incorporated herein by reference.

Lipid-polynucleotide or liposome-polynucleotide formulations suitable for lipofection according to the present invention may be prepared by adding the appropriate amount of polynucleotide to a lipid or liposome solution. In one embodiment, the ratio of polynucleotide to lipid may be in the range of 80 ng to 320 ng poynulceotide per 0.4 μl to 1 μl lipid for transfection of 1×10⁴ cells. Based on these ratios those skilled in the art will be able to determine the appropriate ratio of DNA to lipid for any given number of cells to be transfected.

Telomerase Catalytic Subunit-Encoding Polynucleotides

The present invention contemplates the transfection of collagen-producing cells with a polynucleotide encoding a telomerase catalytic subunit.

Accordingly, aspects of the present invention provide methods for introducing at least one polynucleotide encoding one or more telomerase catalytic subunits into a collagen-producing cell. Typically the polynucleotide is transfected with a lipid-based delivery agent. The present invention also provides isolated collagen-producing cells transfected with at least one polynucleotide encoding one or more telomerase catalytic subunits.

The polynucleotide encoding the one or more telomerase catalytic subunits may comprise a TERT gene, in particular the human TERT (hTERT) gene. For example the polynucleotide may encode a polypeptide comprising an amino acid sequence as set forth in SEQ ID NO:1. The polynucleotide may comprise any nucleotide sequence as set forth in SEQ ID NO:2. The present invention also contemplates the use of homologues of hTERT. Homologues include hTERT-encoding polynucleotides from non-human species. The TERT polynucleotide may be natural, recombinant or synthetic and may be obtained by purification from a suitable source or produced by standard recombinant DNA techniques such as those well known to persons skilled in the art, and described in, for example, Sambrook et al., Molecular Cloning: a Laboratory Manual, Cold Spring Harbor Laboratory Press (the disclosure of which is incorporated herein by reference).

Those skilled in the art will appreciate that the precise sequence of a polynucleotide encoding a telomerase catalytic subunit used according to the methods and compositions of the present invention may vary depending on a number of factors, for example the species of animal to be treated such that the sequence is selected so as to be derived from the species to be treated.

In a particular embodiment, the nucleotide sequence of the hTERT polynucleotide is as set forth in SEQ ID NO:2 or a fragment or variant thereof, or displays sufficient sequence identity thereto to hybridise to the sequence of SEQ ID NO:2. In alternative embodiments, the nucleotide sequence of the polynucleotide may share at least 60%, 70%, 80%, 85%, 90%, 96%, 97%, 98% or 99% identity with the sequence set forth in SEQ ID NO:2.

The polynucleotide encoding hTERT may encode a polypeptide having the amino acid sequence as set forth in SEQ ID NO:1. Within the scope of the term “polypeptide” as used herein are fragments and variants thereof.

The term “fragment” refers to a nucleic acid or polypeptide sequence that encodes a constituent or is a constituent of a full-length sequence. In terms of a TERT polypeptide, the fragment typically possesses qualitative biological activity in common with the full-length TERT.

The term “variant” as used herein refers to substantially similar sequences. Generally, nucleic acid sequence variants encode polypeptides which possess qualitative biological activity in common. Generally, polypeptide sequence variants also possess qualitative biological activity in common. Further, these polypeptide sequence variants may share at least 60%, 65%, 70%, 75%, 80%, 85%, 90%, 95%, 96%, 97%, 98% or 99% sequence identity.

Those skilled in the art will also readily appreciate that in accordance with the present invention various modifications may be made to the sequences of TERT polynucleotides, for example via the insertion or deletion of one or more codons, such that modified variants of the TERT polypeptide are generated. Such modifications are also included within the scope of the term “variant”. For example, modifications may be made so as to enhance the biological activity or expression level of TERT or to otherwise increase the effectiveness of the polypeptide to achieve the desired result. The term “variant” also includes “analogues”, wherein the term “analogue” means a polypeptide which is a derivative of TERT, which derivative comprises addition, deletion, substitution of one or more amino acids, such that the polypeptide retains substantially the same function as native TERT. The term “conservative amino acid substitution” refers to a substitution or replacement of one amino acid for another amino acid with similar properties within a polypeptide chain (primary sequence of a protein). For example, the substitution of the charged amino acid glutamic acid (Glu) for the similarly charged amino acid aspartic acid (Asp) would be a conservative amino acid substitution.

As described above, the TERT polynucleotide to be transfected is typically operably linked to one or more regulatory sequences, for example a suitable promoter, to ensure expression of the encoded polypeptide following transfection.

A variety of promoters suitable for driving expression of exogenous sequences in eukaryotic cells are known to those skilled in the art. Suitable promoters may or may not be telomerase-specific and may be constitutive, inducible or repressible. Inducible and repressible promoters are particularly advantageous if it is desired to regulate, for example temporally, the expression of the telomerase catalytic subunit. Inducible systems typically operate by adding to the cell containing the transfected vector one or more agents to activate the promoter and switch on expression of the linked polynucleotide. Inducible promoter systems typically make use of a chemical inducer, either added exogenously or being an expressed gene product to bind to a target sequence, thereby activating the promoter. In the absence of the inducer, the promoter is inactive and the polynucleotide(s) operably linked thereto are not expressed. For example, inducible systems include metal-responsive promoters, steroid-regulated promoters and tetracycline-regulated promoters. Repressible systems typically operate by adding to the cell containing the transfected vector one or more agents to repress the promoter thereby switching off expression of the linked polynucleotide in the presence of the agent.

The vector may be episomal such that the TERT polynucleotide contained therein is maintained extra-chromosomally, thereby eliminating any potential detrimental effects of chromosomal integration. Alternatively, the vector may enable or facilitate integration of the TERT polynucleotide into the genome. The choice of an episomal or integrating vector will depend largely on the particular circumstances, for example the cell type to be transfected and whether is transient or long term expression of TERT is desired.

The present invention contemplates the transfection of polynucleotides encoding telomerase catalytic subunits either alone or in combination with any other polynucleotides. For example it may be desirable to co-transfect a polynucleotide encoding a telomerase catalytic subunit with a polynucleotide encoding or comprising a telomerase RNA moiety and/or one or more anabolic growth factors or other signal transduction molecules. Suitable growth factors may include OP-1, MSX and BMP. In such embodiments, polynucleotides may be located on separate nucleic acid constructs or on the same construct. In embodiments in which the polynucleotides are located on the same construct, they may be operably linked to the same or different promoters. Further, an anabolic effect may be achieved by co-administering one or more antioxidants such as nitric oxide and/or antimetabolites to the cells.

Collagen-Producing Cell Defects and Intervertebral Disc Degeneration

The present invention also provides methods and compositions for treating patients having conditions or diseases arising from defects in collagen-producing cells, and to methods and compositions for preventing such conditions or diseases.

For example, transfection of intervertebral disc cells using polynucleotides encoding telomerase catalytic subunits according to methods of the invention finds application in the treatment of intervertebral disc degeneration or the prevention of intervertebral disc degeneration. Treating may comprise slowing, fully or partially inhibiting, or otherwise retarding the progression of the degeneration process or reversing the degeneration process. Prevention may prevent the onset or establishment of the degeneration process in individuals considered susceptible thereto.

Examples of conditions and diseases associated with intervertebral disc degeneration to which the present invention finds application include, but are not limited to, discogenic axial pain including back and neck pain, foraminal stenosis, malalignment, spinal segmental instability, spinal stenosis, and disc herniations such as herniations of the nucleus pulposus.

In transfecting chondrocytes according to methods of the present invention, the invention finds application in tendon healing and the treatment of osteochondral defects and injuries such as rotator cuff tears of the shoulder, tennis elbow, and osteochondral defects of the knees and ankles.

In particular embodiments of the present invention treatments according to the present invention are typically achieved using ex vivo procedures. For example, in application to the treatment of intervertebral disc degeneration or axial pain in individuals, cells would typically be removed from an individual, transfected according to a method of the present invention and re-introduced into the one or more intervertebral discs of the individual in need of treatment. The cells to be transfected may be autologous. The cells may be intervertebral disc cells, for example removed during a discography procedure, may be other cartilage cells or collagen-producing cells including fibroblasts, or may be derived from any potential donor region including, for example, the skin, subcutaneous tissue, fat, muscles or rib cartilage. In alternative embodiments the cells to be transfected ex vivo may be allogeneic being procured from either cadaveric or live human donors.

Ex vivo cell therapy could also be employed using mesenchymal stem cells taken either from bone marrow or adult peripheral blood, embryonic stem cells, any other pluripotent or totipotent cells, or ‘designer’ cells generated in vitro. In alternative embodiments cellular component(s) of a tissue engineered device to replace part(s) of intervertebral disc are transfected with the said polynucleotide to enhance extracellular matrix production and/or prolong cellular viability within the tissue engineered product. The tissue engineered product may be used, for example, to replace or heal parts of the intervertebral disc or cartilage in treating osteochondral defects, or tendon for rotator cuff tears, or the meniscus of the knee.

Surgical delivery of transfected cells to an individual in need thereof may be achieved by various methods known to those skilled in the art. For example, transfected cells may be directly implanted into the region of interest in the patient by way of an infusion pump for continuous infusion. Alternatively, the transfected cells may be associated with a locally implantable device which, for example, replaces part of the nucleus region of an intervertebral disc or any adjacent anatomical structure.

Single or multiple doses may be required to effectively treat the condition or achieve the desired effect. For example, in the case of ex vivo cell manipulations followed by surgical delivery of transfected cells, multiple transfections of cells may be carried out and/or multiple administrations of transfected cells may be required. It will be clear to those skilled in the art that the optimal amount of cells to be administered and the optimal number of administrations for the effective treatment of a particular condition may be determined on a case by case basis. Similarly, in any given circumstance the optimal amount of DNA to be transfected into cells, whether ex vivo or in vivo, to generate the desired effect may be determined by those skilled in the art.

Treatments according to the present invention may be administered in combination with other therapies for the treatment or prevention of conditions or injuries associated with intervertebral disc degeneration. For example, treatments of the invention may be used in combination with other agents known to assist in the reduction or prevention of intervertebral disc degeneration or may be administered in combination with other surgical procedures for intervertebral disc restoration or regeneration (such as are described in Diwan, A. D. et al. 2000, Current concepts in intervertebral disk restoration, Orthop Clin N Am 31:453-464).

Compositions

Polynucleotides encoding telomerase catalytic subunits may be administered in the form of a composition, together with one or more pharmaceutically acceptable carriers and optionally, a lipid-based delivery agent. Compositions may be administered for either therapeutic or preventative purposes. In a therapeutic application, compositions are administered to a patient already suffering from a disorder or injury. In therapeutic applications the treatment may be for the duration of the disease state.

The therapeutically effective dose level for any particular patient will depend upon a variety of factors including: the disorder being treated and the severity of the disorder or injury; activity of the compound or agent employed; the composition employed; the age, body weight, general health, sex and diet of the patient; the time of administration; the route of administration; the rate of sequestration of the agent or compound; the duration of the treatment; drugs used in combination or coincidental with the treatment, together with other related factors well known in medicine.

One skilled in the art would be able, by routine experimentation, to determine an effective, non-toxic amount of agent or compound which would be required to treat applicable diseases.

Further, it will be apparent to one of ordinary skill in the art that the optimal quantity and spacing of individual dosages will be determined by the nature and extent of the disease state or injury being treated, the form, route and site of administration, and the nature of the particular individual being treated. Also, such optimum conditions can be determined by conventional techniques.

It will also be apparent to one of ordinary skill in the art that the optimal course of treatment, such as, the number of doses of the composition given per day for a defined number of days, can be ascertained by those skilled in the art using conventional course of treatment determination tests.

In general, suitable compositions may be prepared according to methods Which are known to those of ordinary skill in the art and accordingly may include a pharmaceutically acceptable carrier, diluent and/or adjuvant. The carriers, diluents and adjuvants must be “acceptable” in terms of being compatible with the other ingredients of the composition, and not deleterious to the recipient thereof.

Examples of pharmaceutically acceptable carriers or diluents are demineralised or distilled water; saline solution; vegetable based oils such as peanut oil, safflower oil, olive oil, cottonseed oil, maize oil, sesame oils such as peanut oil, safflower oil, olive oil, cottonseed oil, maize oil, sesame oil, arachis oil or coconut oil; silicone oils, including polysiloxanes, such as methyl polysiloxane, phenyl polysiloxane and methylphenyl polysolpoxane; volatile silicones; mineral oils such as liquid paraffin, soft paraffin or squalane; cellulose derivatives such as methyl cellulose, ethyl cellulose, carboxymethylcellulose, sodium carboxymethylcellulose or hydroxypropylmethylcellulose; lower alkanols, for example ethanol or iso-propanol; lower aralkanols; lower polyalkylene glycols or lower alkylene glycols, for example polyethylene glycol, polypropylene glycol, ethylene glycol, propylene glycol, 1,3-butylene glycol or glycerin; fatty acid esters such as isopropyl palmitate, isopropyl myristate or ethyl oleate; polyvinylpyrolidone; agar; gum tragacanth or gum acacia, and petroleum jelly. Typically, the carrier or carriers will form from 10% to 99.9% by weight of the compositions.

Compositions can be administered by standard routes. In general, the compositions may be administered by the parenteral route, that is intraspinal, subcutaneous, intramuscular or intravenous.

For administration as an injectable solution or suspension, non-toxic parenterally acceptable diluents or carriers can include, Ringer's solution, isotonic saline, phosphate buffered saline, ethanol and 1,2 propylene glycol. Methods for preparing parenterally administrable compositions are apparent to those skilled in the art, and are described in more detail in, for example, Remington's Pharmaceutical Science, 15th ed., Mack Publishing Company, Easton, Pa., hereby incorporated by reference.

Kits

In accordance with the present invention, kits containing polynucleotide(s) encoding telomerase catalytic subunit(s) together with lipid-based delivery agents for the transfection of such polynucleotide(s) into collagen-producing cells may be prepared. Such kits may be used in accordance with the methods of the present invention, for example, in increasing collagen-expression in collagen-producing cells, increasing extracellular matrix production in collagen-producing cells, or treating impaired collagen-producing cell activity, intervertebral disc degeneration or axial pain associated with intervertebral disc degeneration, in individuals in need of such treatment.

Kits according to the present invention may also include other components required to conduct the methods of the present invention, such as buffers and/or diluents. The kits typically include containers for housing the various components and instructions for using the kit components in the methods of the present invention.

The reference in this specification to any prior publication (or information derived from it), or to any matter which is known, is not, and should not be taken as, an acknowledgment or admission or any form of suggestion that that prior publication (or information derived from it) or known matter forms part of the common general knowledge in the field of endeavour to which this specification relates.

The present invention will now be further described in greater detail by reference to the following specific examples, which should not be construed as in any way limiting the scope of the invention.

### EXAMPLES

### Example 1

### Transfection of Sheep Intervertebral Disc Cells in the Presence of Lipofectamine

The optimal ratio of DNA to Lipofectamine 2000 for transfection of sheep intervertebral disc cells in vitro was determined. Annulus fibrosus cells were isolated from fresh cadaveric sheep spine (from a 2 year old sheep) and cultured till confluency following overnight collagenase (0.025%) digestion at 37° C. Isolated cells were grown in 1% fetal calf serum in DMEM and in the presence of 1% antibiotics. Passage three cells were seeded into 96 well plates for transfection efficiency studies.

Cells were seeded at 1×10⁴ per well and transfected one day post seeding. The DNA transfected was a reporter gene (pCMV.SPORT-βgal, Invitrogen), allowing the fate of the transfected DNA to be traced. The concentrations of DNA tested were 80 ng, 160 ng, 240 ng and 320 ng. The concentrations of Lipofectamine 2000 reagent (Invitrogen) tested against the different DNA concentrations were 0.4 μl, 0.6 μl, 0.8 μl and 1 μl. The transfection protocol followed manufacturer's instructions. β-galactosidase activity was tested 24 hours post transfection to determine which combinations of DNA and Lipofectamine 2000 were most efficient in DNA transfection. Untransfected cells were used as a negative control.

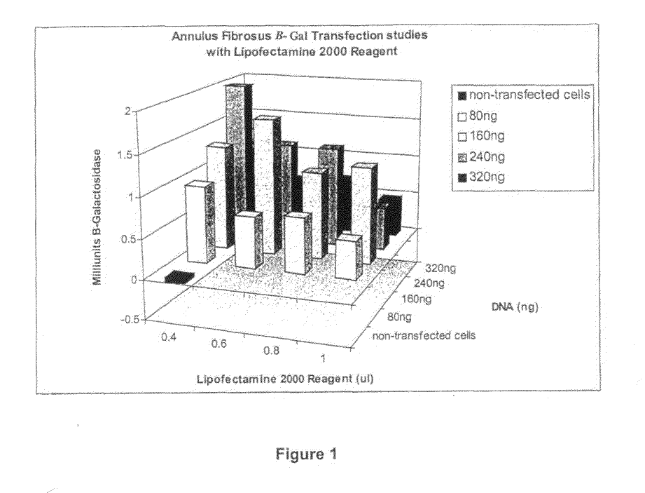

The results of transfection studies are shown in FIG. 1. Transfection efficiency was determined to be optimal at 240 ng DNA per 0.4 μl Lipofectamine 2000 followed by 160 ng DNA per 0.6 μl Lipofectamine 2000. These ratios were then used for future transfection studies in sheep intervertebral disc cells in culture as described in the following examples.

### Example 2

### Telomerase Expression and Telomere Length in Sheep Intervertebral Disc Cells Transfected with hTERT

Sheep nucleus pulposus cells were transfected using the conditions described in Example 1, using 240 ng of vector DNA containing the hTERT (human telomerase reverse transcriptase) gene in 0.6 μl of Lipofectamine 2000. The plasmid (pCI-neo from Promega) containing the hEST2 sequence (from positions 51 to 3456) cloned into the EcoRI-SalI restriction sites was supplied by Roger Reddel from the Childrens Medical Research Institute at Westmead Hospital, Sydney, Australia (Colgin et al. 2000, The hTERT α splice variant is a dominant negative inhibitor of telomerase activity. Neoplasia, 2:426-432). Transfection proceeded for 24 hours and the cells were harvested for analysis using the telomeric repeat amplification protocol (TRAP) assay.

The telomerase activity assay [TeloTAGGG Telomerase PCR ELISA^{PLUS} (TRAP), Roche] was conducted according to manufacturer's instructions, whereby cells were lysed to release telomerase enzyme. Released enzyme is then detected by its ability to elongate synthetic telomere DNA in vitro. The newly synthesised stretch of telomeric DNA is PCR amplified for detection by ELISA immunoassay for quantitative determination of telomerase activity.

Briefly, for the TRAP assay the telomerase substrate is biotinylated, enabling its binding to steptavidin-coated wells of a 96 well plate. The probe for detecting the telomeric repeat sequences is digoxigenin (DIG)-labeled, and is detected using an anti-DIG antibody conjugated to horseradish peroxidase (Anti-DIG-HRP). Binding is detected by colourimetric reaction using the substrate for horseradish peroxidase tetramethylbenzidine and measuring absorbance.

For relative telomerase activity (RTA) determination, absorbance readings were taken at 450 nm and the background (A690 nm) subtracted.

The use of internal standards allowed for the standardization of the activity detected between different tubes due to variances other than the transfected vectors that may have been introduced during the PCR amplification or ELISA. The internal standards were DNA at a known concentration of 0.001 amol/μg. The internal standards were in the same reaction tubes as the samples enabling the detection of any inhibitors of the amplification process that would lead to a misinterpretation of the results.

Prior to analysis of results, the negative control must have an absorbance of A450 nm minus background A690 nm (A450 nm−A690 nm) of less than 0.1. The ‘difference in absorbency’ for the negative control (heat inactivated hTERT-containing_samples) was 0.064 (A450 nm-A690 nm) units, confirming the validity of the activities observed for the test samples. Samples were deemed positive for telomerase activity if the ‘difference in absorbency’ ((A450 nm-A690 nm) for the sample minus (A450 nm-A690 nm) for the negative control) was twice that of the blank.

hTERT sample 1: 0.357−0.064 (Ab. samples−Ab. negative controls)=0.293

hTERT sample 2: 0.370−0.064 (Ab. samples−Ab. negative controls)=0.306

Background value multiply by two to get the two times value:

hTERT A690 nm: 0.042×2=0.084

hTERT A690 nm: 0.032×2=0.070

The telomerase activity results are shown in Table 1 below.

TABLE 1
Telomerase activities

| | TELOMERASE | INTERNAL |
|---|---|---|
| SAMPLES | ACTIVITY | STANDARD |
| hTERT | 0.357 | 1.052 |
| hTERT | 0.370 | 1.025 |
| CONTROL (vector only) | 0.045 | 0.982 |
| CONTROL (vector only) | 0.032 | 0.982 |
| −ve control (heat inactivated | 0.064 | |
| hTERT samples) | | |
| +ve control (known concentration, | 0.108 | 0.968 |
| low activity) | | |
| +ve control (known concentration, | 2.373 | 0.968 |
| high activity) | | |
| −ve control (lysis buffer only, −ve | 0.027 | |
| control for the kit) | | |

Values for each of the hTERT-containing samples are at least twice their respective background values.

The figures from Table 1 were used in the following formula to determine RTA:

$$RTA = \frac{( {A}_{S} - {A}_{SO} ) / {A}_{S , I S}}{( {A}_{TS 8} - {A}_{TS 8 , O} ) / {A}_{TS 8 , I S}} \times 100$$

where,
A_{S}=absorbance of samples
A_{SO}=absorbance of negative controls
A_{SIS}=absorbance Internal Standards (IS)
A_{TS8}=absorbance Control template (TS8)
A_{TS80}=absorbance of lysis buffer
A_{TS81S}=absorbance of Internal Standard of control template

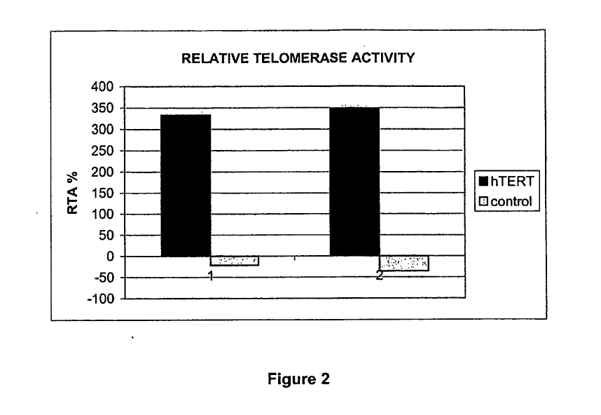

RTA values are shown in FIG. 2. As shown in Table 1 and FIG. 2 transfection was demonstrated to be successful and telomerase activity 24 hours post transfection was achieved in cells transfected with the hTERT gene. No telomerase activity was detected in cells transfected with control vector (−hTERT).

The TeloTAGGG Telomere Length Assay kit (Roche) was subsequently used to determine the length of telomeres 24 hours, 73, 278 and 329 days following transfection of the sheep nucleus pulposus cells with the hTERT gene. Genomic DNA was isolated from transfected cells and Hinfl/RsaI restriction digestion performed according to manufacturer's instructions to remove all non-telomeric DNA. Southern blot detection was then used to detect telomere length.

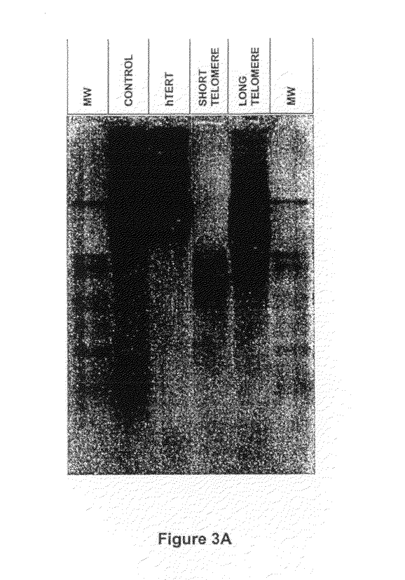

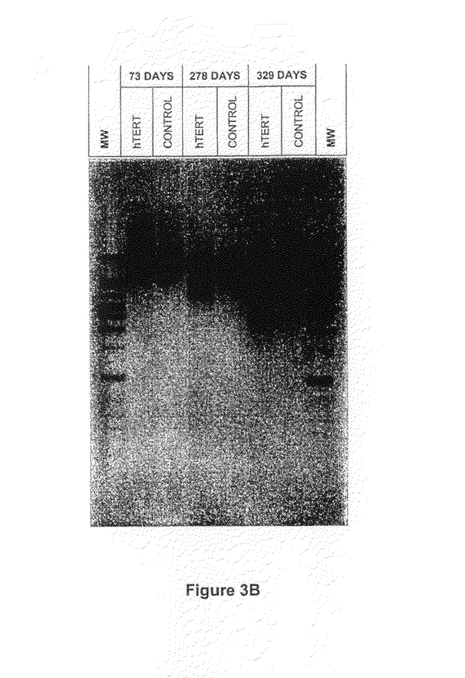

As illustrated in FIG. 3, telomeres isolated from cells transfected with hTERT were not significantly different in length to those isolated from cells transfected with vector only (−hTERT). The sheep cells have relatively long telomeres even only 24 hours post-transfection and extension was not evident. The beneficial effects of telomerase in the sheep nucleus pulposus cells is therefore shown to be independent of the actual telomere length.

### Example 3

### Population Doubling in Sheep Intervertebral Disc Cells Transfected with hTERT

Sheep spinal cells were extracted following the procurement of fresh cadaveric sheep spines. The two regions in the disc, annulus fibrosus (outer region) and the nucleus pulposus (inner region) were visually separated. The nucleus pulposus cells were treated with collagenase overnight and cultured. Following several passages these cells were transfected with a vector containing the hTERT gene using Lipofectamine 2000 (Invitrogen) as described above. 2 days post transfection the transfected cells were selected using neomycin selective marker, antibiotic G418 (Invitrogen) for at least two weeks (according to manufacturer's instructions). Six flasks were set up each for hTERT-transected and vector only-transected cells. On confluency, cells were trypsinised, counted and reseeded with 6×10⁵ cells/flask. Data from all six flasks was averaged and the population doubling was then calculated by the formula: PD=log(cell output/cell input)/log 2. Cumulative population doubling was plotted against time with mean±SEM and statistical significance determined by student's t-test with equal variances.

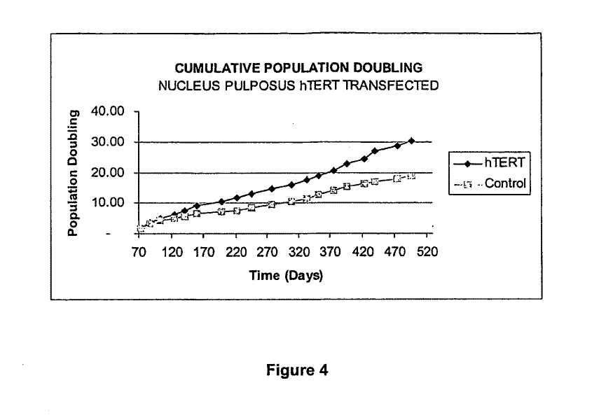

A significant difference in population doubling was observed between the hTERT- and vector-transfected cells (FIG. 4). Following 160 days post transfection the difference in cumulative population doubling between hTERT- and vector-transfected cells was statistically significant (t-test; P<0.05).

### Example 4

### Real Time PCR Analysis of Collagen Expression in Sheep Intervertebral Disc Cells Transfected with hTERT

Real time PCR was used to determine the level of expression of type I and type II collagen and aggrecan in sheep intervertebral disc cells. Cells were transfected with a vector containing hTERT in the presence of Lipofectamine 2000, as described above.

Primers for Collagen type-I and type-II, the proteoglycan aggrecan and glyceraldehyde-3-phosphate dehydrogenase (GAPDH) sequences were designed using Primer3 online software and obtained from Invitrogen. The primer sequences are as follows:

| | |
|---|---|
| Collagen II: | |
| (F) AACACTGCCAACGTCCAGATG | (SEQ ID NO: 3) |
| (R) TCGTCCAGATAGGCAATGCTG | (SEQ ID NO: 4) |
| Collagen I: | |
| (F) AGACATCCCACCAATCACCT | (SEQ ID NO: 5) |
| (R) AGATCACGTCATCGCACAAC | (SEQ ID NO: 6) |
| Aggrecan: | |
| (F) ACGTGATCCTCACGGCAAA | (SEQ ID NO: 7) |
| (R) GTGAAAGGCTCCTCAGGTTCTG | (SEQ ID NO: 8) |
| GAPDH: | |
| (F) ACCCAGAAGACTGTGGATGG | (SEQ ID NO: 9) |
| (R) AGAGGCAGGGATGATGTTCT | (SEQ ID NO: 10) |

RNA was isolated using an RNeasy RNA isolation kit (Promega) from cells transfected with hTERT and those transfected only with vector. cDNA was then produced following DNase digestion of the isolated RNA. Real time PCR was carried out using a Corbett Research thermal cycler and amplified product detected by Syber Green (Invitrogen) staining. Touchdown real-time PCR was performed with 50 cycles of (denaturation at 94° C.; 30 seconds, annealing at 60° C.; 30 seconds and elongation at 73° C.; 60 seconds. The primers were used at 5 μM per 25 μL reaction. Cycle times (Ct) and amplification efficiencies (E) of each gene were obtained from the experimental data given by the thermal cycler. Analysis of relative gene expression in hTERT transfected cells compared to vector only cells was performed using REST© (Relative Expresion Software Tool) (see Table 2 below). All cycle times were normalized with the endogenous housekeeping gene (GAPDH).

TABLE 2
Relative expression of collagen type I, collagen type II and
aggrecan in hTERT-transfected cells
hTERT MATRIX EXPRESSION RELATIVE TO CONTROL

| Days | Collagen 1 | p-value | Collagen 2 | p-value | Aggrecan | p-value |
|---|---|---|---|---|---|---|
| 87 | −9.664 | 0.077 | −3.247 | 0.011 | 2.165 | 0.077 |
| 142 | 8.713 | 0.001 | 2.135 | 0.001 | −1.608 | 0.147 |
| 246 | 5.924 | 0.001 | 2.175 | 0.001 | −1.288 | 0.034 |
| 309 | 8.590 | 0.001 | 2.000* | N/A | 1.195 | 0.472 |
| 376 | 23.612 | 0.001 | 23.036 | 0.001 | −3.280 | 0.001 |
| 424 | 46.074 | 0.001 | 32.434 | 0.001 | −2.837 | 0.064 |

*Derived from trend line due to loss of sample
Figures in bold type represent statistically significant results In comparison with controls

The figures in Table 2 were calculated using the primer (target) PCR efficiencies (E) and cycle times (CP) of a certain fluorescence threshold in the real time run normalized to GAPDH (ref) according to the following formula derived from Pfaffle, M. W. 2001, A new mathematical model for relative quantification in real-time RT-PCR, Nucl Acid Res 29:e45:

Ratio=(E_{target})^{ΔCPtarget(control-sample)}/(Eref)^{ΔCPref(control-sample)}

This calculation provides quantification of matrix expression in the hTERT-transfected cells (sample) compared to the control cells. The p-values and significance were calculated using the computer program REST (relative expression software tool) available at http://www.wzw.turn.de/gene-quantification/ (Pfaffle. M. W. et al. 2002, Relative expression software tool (REST) for group-wise comparison and statistical analysis of relative expression results in real-time PCR, Nucl Acid Res 30:e36).

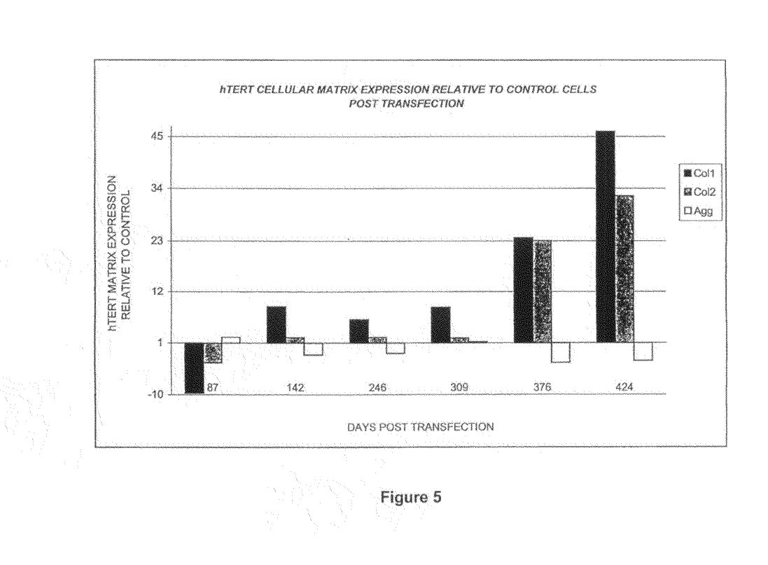

As illustrated in FIG. 5, hTERT-transfected cells showed an average of 6-9 fold increase in Collagen type-I mRNA expression compared to vector-transfected cells throughout the experiment (P<0.001). Collagen type-II mRNA expression was doubled in hTERT-transfected cells over that observed in vector-transfected cells. The dramatic increase in expression of both collagen type-I and type-II at sampling days 376 and 424 days post transfection is a reflection of the lack in cellular matrix productivity of vector-only cells on the path of senescence shown by day 439. Although in general aggrecan production by hTERT transfected cells was less than that of vector only cells, the difference observed in most parts were not significant (P<0.001). This is not unusual as chondrocytes are known to dedifferentiate into fibroblast like cells in continual cultures therefore losing characteristics such as proteoglycan production. On day 424 there was no significant difference in aggrecan expression between the two groups even though vector only cells were highly deficient in matrix production shown by the collagen data, suggesting that the overall level of aggrecan was low, if barely detectable, in either samples throughout the entire experiment.

### Example 5

### MTS Cell Proliferation Assay in Sheep Intervertebral Disc Cells Transfected with hTERT

CellTiter 96® AQueous One Solution Cell Proliferation Assay (Promega) was used to measure cell viability by measuring the metabolism of a novel tetrazolium compound 3-(4,5-dimethylthiazol-2-yl)-5-(3-carboxymethoxy-phenyl)-2-(4-sulfonyl)-2H-tetrazolium (MTS) into a water-soluble formazan that absorbs light at 490 nm. Cells transfected as described in previous examples were plated in a 96-well plate, incubated overnight and then incubated in the presence of MTS for 1-4 hours. The absorbency of each sample was then recorded and graphed.

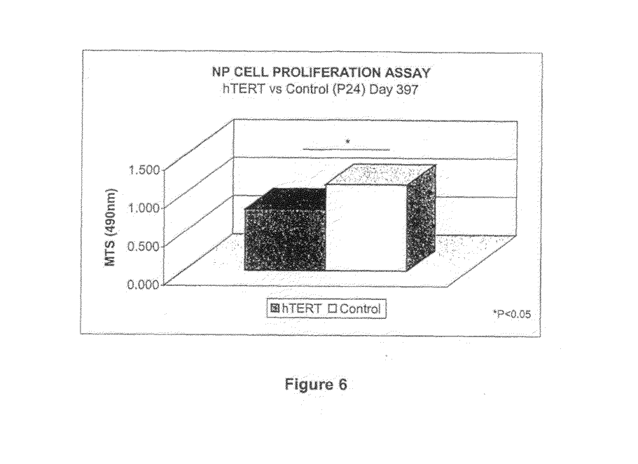

FIG. 6 shows that hTERT-transfected cells have a slower proliferation rate than the vector-transfected cells. Although hTERT-transfected cells were shown to proliferate more slowly, these spindle-like cells had a higher contact inhibition density in which more cells eventually fitted onto the same surface area as that of the rounder vector-only transfected cells on confluency. In addition to their increase in population doubling, this signifies that more, longer living cells can populate an area, such as the intervertebral disc.

### Example 6

### Senescence Assay in Sheep Intervertebral Disc Cells Transfected with hTERT

Senescence Cell Staining Kit (Sigma) was used to detect β-galactosidase activity at pH 6, wherein positive (blue) staining is an indicator of cellular senescence. Cells that had been transfected with either hTERT-containing vector DNA or vector only (as described in previous examples) were seeded in an 8-well chamber glass slide overnight.

The cells at day 439 (passage 26) post transfection were washed twice with PBS and fixed with a formaldehyde/glutaraldehyde solution for 7 minutes. Three subsequent washes were performed to ensure complete removal of fixative. The stain was then added to each well and incubated in a 37° C. incubator in the absence of CO₂ for an optimal staining time of 9 hours. The cells were then counted under a light microscope by 2 independent viewers and the percentage of senescent, blue stained cells determined. Data was arsine transformed prior to a student's t-test for statistical significance.

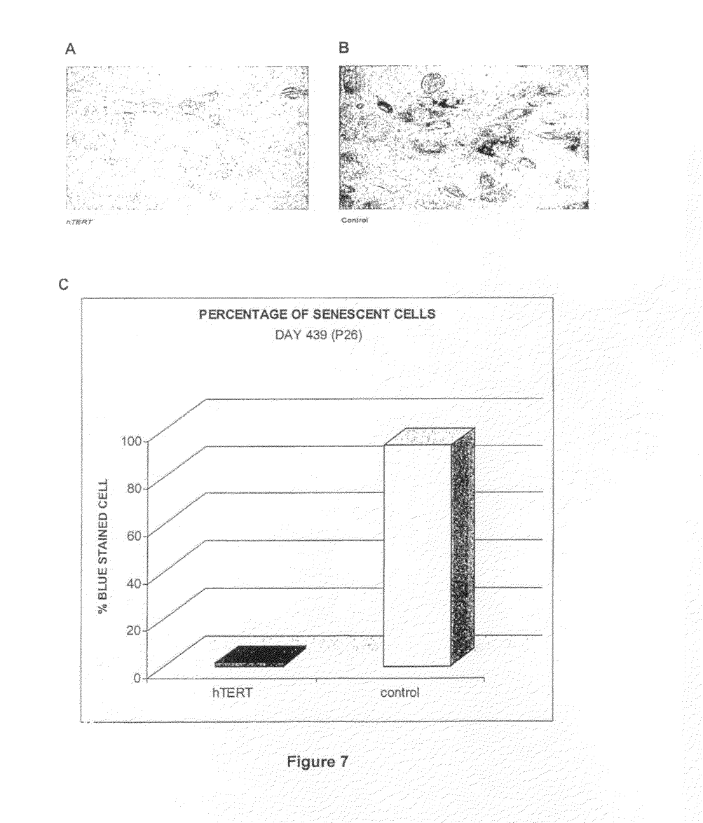

Cells staining positive were detected among the vector-transfected cells (FIG. 7B) at significantly higher frequency than among those cells transfected with hTERT (FIG. 7A). Displayed graphically in FIG. 7C, it can be seen that hTERT-transfected cells (at passage 26, day 439 post transfection) showed no significant level of senescence, whilst more than 80% senescence was observed among vector-transfected cells.

### Example 7

### In Vitro Transformation Analysis

In previous studies, long term cultures of some telomerase immortalized cells have resulted in karyotype instability, inactivation of tumor suppressor genes and even spontaneous tumorigenesis, rendering the risk of hTERT induced immortality being associated with cancer. Accordingly, prior to the use of telomerase gene therapy for treatment of degenerative disease, the present inventors carried out various in vitro transformation studies to address potential carcinogenic risk, including cell cycle functionality following DNA damage and growth of cells in abnormal environments including low serum or in suspension by anchorage-independent means.

For G1 cell cycle checkpoint analysis, cultures were incubated with 7.5 nM actinomycin-D (Sigma-Aldrich) for 24 hours and trypsin-harvested cellular pellets were 70% cold-EtOH fixed overnight. Staining solution (50 μg of propidium iodide, 5% Triton-X100, 1 mg RNase in 1 ml PBS) was applied one hour prior to flow cytometry. Data was analyzed using Cylchred software (Cardiff University, Wales, United Kingdom). p53 protein levels were also analysed following incubation of cells in 7.5 nM actinomycin-D for 24 hours. Homogenization buffer (50 mM Tris pH 7.4, 0.1 mM EDTA, Leupeptin 1 μg/ml, Pepstatin 5 μg/ml, AEBSF 200 μg/ml (Sigma-Aldrich)) was added directly to the cultures for 10 min on ice prior to cellular removal with a cell scraper. Cellular lysates were briefly sonicated and the protein concentration determined using the Micro BCA™ Protein Assay Kit (Pierce, Rockford, Ill.). 25 μg protein extract was separated on 12% SDS-polyacrylamide gels. Proteins were transferred to PolyScreen® PVDF hybridization membranes (PerkinElmer, Wellesley, Mass.). p53 and β-actin was probed with mouse monoclonal; anti-p53 (Ab-3) (OP29, Calbiochem, La Jolla, Calif.) and anti-β-actin (Chemicon, Temecula, Calif.) primary antibodies respectively, labeled with horseradish peroxidase-conjugated secondary antibodies (Chemicon), the complexes were detected by the Super Signal Chemilumnescent Substrate system (Pierce).

For determination of anchorage independent growth triplicate plates of 1×10⁴ cells were seeded in 2 ml of (0.4% Bacto™ Agar (BD, Franklin Lakes, N.J.), 24% FCS in DMEM) over 1 ml of solidified 0.8% Bacto™ Agar in a 35 mm diameter dish.

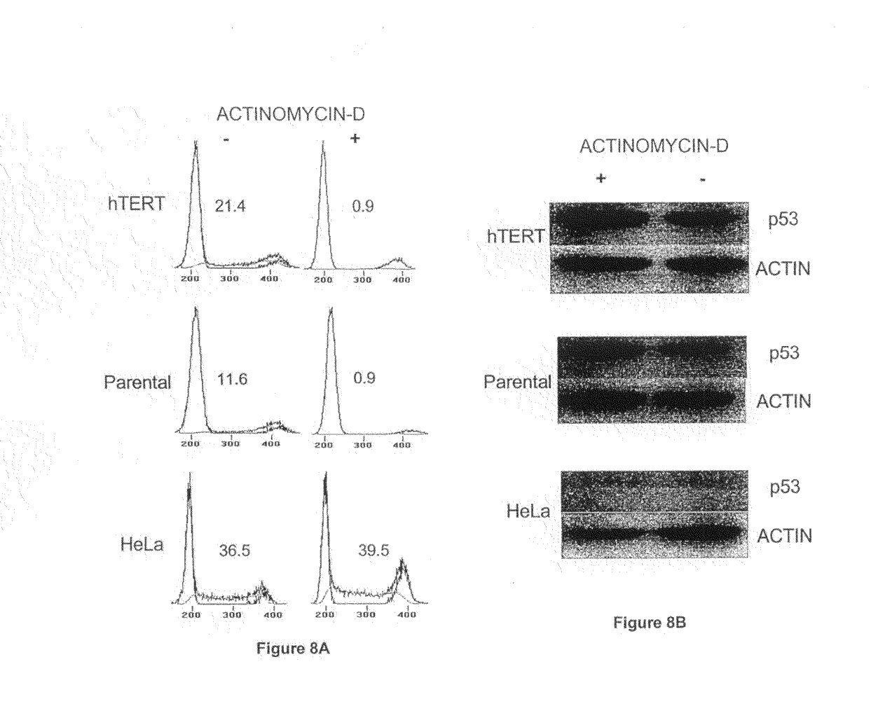

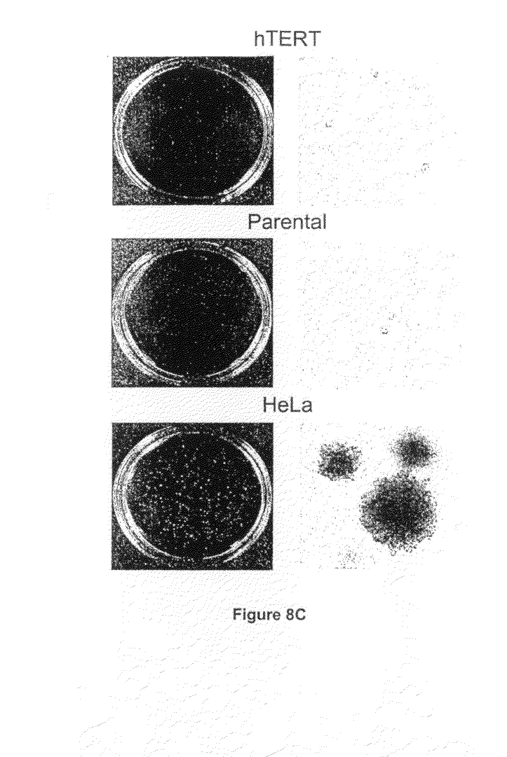

In-vitro transformation properties were tested on one of the six hTERT-transfected flasks with the highest population doublings. Due to the senescence of control cells, early passage untransfected (parental) nucleus pulposus cells were used as negative controls and HeLa cells as positive controls. Following actinomycin-D damage, the growth phase of HeLa cultures was not hindered whilst both parental and hTERT-transfected cells ceased growth with only 0.9% of cells remaining in S-phase compared to the 11.6% and 21.4%, respectively, in the S-phase of normal cultures (see FIG. 8A). Correlating with this data, HeLa cells failed to produce sufficient p53 expression in response to actinomycin-D while similar levels of p53 protein expression was produced in parental and hTERT-transfected cells (FIG. 8B). β-actin expression was used as a positive control.

Neoplastic transformation properties of anchorage independent or serum starvation growth was not evident in hTERT-transfected cells. Growth of agar suspension cultures were seen only in HeLa cells with no colony formation observed for either parental or hTERT-transfected cells over three weeks of culture (FIG. 8C). Further, in serum starvation conditions (triplicate cultures maintained in DMEM with 0.2% FCS, seeded at sub-confluent densities and continually passaged on confluency with cumulative population doubling determined for 38 days), HeLa cells showed continued growth while parental cells experienced a short growth phase and hTERT-transfected cells had no growth (data not shown).

1. A method for introducing at least one polynucleotide of interest into an intervertebral disc cell, wherein the polynucleotide of interest is transfected with a lipid-based delivery agent.

2. The method of claim 1 wherein the intervertebral disc cell is a cell of the nucleus pulposus or annulus fibrosus.

3-110. (canceled)

111. The method of claim 1 wherein the intervertebral disc cell is a fibroblast, chondroblast or chondrocyte.

112. The method of claim 1 wherein the lipid based delivery agent is a cationic lipid.

113. The method of claim 112 wherein the cationic lipid is Lipofectamine 2000.

114. The method of claim 1 wherein the polynucleotide encodes one or more telomerase catalytic subunits.

115. The method of claim 114 wherein the telomerase catalytic subunit is the telomerase reverse transcriptase encoded by the TERT gene.

116. The method of claim 115 wherein the telomerase reverse transcriptase comprises the amino acid sequence as set forth in SEQ ID NO:1.

117. The method of claim 115 wherein the TERT gene comprises the nucleotide sequence as set forth in SEQ ID NO:2.

118. A method for introducing at least one polynucleotide encoding one or more telomerase catalytic subunits into an intervertebral disc cell, wherein the polynucleotide is transfected with a lipid-based delivery agent.

119. An isolated intervertebral disc cell transfected with at least one polynucleotide encoding one or more telomerase catalytic subunits.

120. The cell of claim 119 wherein the telomerase catalytic subunit is the telomerase reverse transcriptase.

121. The cell of claim 119 wherein the polynucleotide is transfected in the presence of a lipid-based delivery agent.

122. The cell of claim 121 wherein the lipid based delivery agent is a cationic lipid.

123. The cell of claim 122 wherein the cationic lipid is Lipofectamine 2000.

124. The cell of claim 119 wherein the cell displays elevated collagen expression when compared with an intervertebral disc cell not transfected with the at least one polynucleotide.

125. A method for increasing collagen expression in a collagen-producing cell, the method comprising introducing into the cell or a progenitor thereof at least one polynucleotide encoding one or more telomerase catalytic subunits.

126. The method of claim 125 wherein the collagen-producing cell is an intervertebral disc cell.

127. The method of claim 125 wherein the polynucleotide is transfected in the presence of a lipid-based delivery agent.

128. The method of claim 125 wherein the telomerase catalytic subunit is the telomerase reverse transcriptase encoded by the TERT gene.

129. The method of claim 125 wherein the polynucleotide(s) is introduced into the cells ex vivo and the transfected cells are introduced into a patient.

130. A method for increasing extracellular matrix formation by a collagen-producing cell, the method comprising introducing into the cell or a progenitor thereof at least one polynucleotide encoding one or more telomerase catalytic subunits.

131. The method of claim 130 wherein the collagen-producing cell is an intervertebral disc cell.

132. The method of claim 130 wherein the polynucleotide is transfected in the presence of a lipid-based delivery agent.

133. The method of claim 130 wherein the telomerase catalytic subunit is the telomerase reverse transcriptase encoded by the TERT gene.

134. The method of claim 130 wherein the polynucleotide(s) is transfected into the cells ex vivo and the transfected cells are introduced into a patient.

135. A method for increasing the life span of an intervertebral disc cell, the method comprising introducing into the cell or an immediate progenitor thereof at least one polynucleotide encoding one or more telomerase catalytic subunits.

136. A method for treating or preventing a condition associated with impaired collagen production in collagen-producing cells, the method comprising administering to an individual an effective amount of at least one polynucleotide encoding one or more telomerase catalytic subunits, wherein said administration results in an increase in collagen production by said collagen-producing cells.

137. The method of claim 136 wherein the polynucleotide(s) is introduced into one or more collagen-producing cells ex vivo and the transfected cells are introduced into the individual.

138. A method for treating or preventing intervertebral disc degeneration, or a condition or disease associated with intervertebral disc degeneration in an individual, the method comprising administering to the individual an effective amount of at least one polynucleotide encoding one or more telomerase catalytic subunits.

139. The method of claim 139 wherein the polynucleotide(s) is introduced into one or more collagen-producing cells ex vivo and the transfected cells are introduced into the patient.

140. The method of claim 139 wherein the polynucleotide is transfected into the cellular subunit of a tissue engineered component to replace one or more parts of the intervertebral disc.

141. The method of claim 139 wherein the condition is axial pain.

142. The method of claim 141 wherein the axial pain is back and neck pain.

143. A composition for treating or preventing a condition associated with impaired collagen production, intervertebral disc degeneration, or a condition or disease associated with intervetebral disc degeneration, the composition comprising at least one polynucleotide encoding one or more telomerase catalytic subunits together with a lipid-based delivery agent.


SEQUENCE LISTING (10 sequences)

SEQ ID NO: 1 - protein, 1132 aa, Homo sapiens [sequence omitted]

SEQ ID NO: 2 - DNA, 3399 bp, Homo sapiens
atgccgcgcgctccccgctgccgagccgtgcgctccctgctgcgcagccactaccgcgaggtgctgccgctggccacgttcgtgcggcgcctggggccccagggctggcggctggtgcagcgcggggacccggcggctttccgcgcgctggtggcccagtgcctggtgtgcgtgccctgggacgcacggccgccccccgccgccccctccttccgccaggtgtcctgcctgaaggagctggtggcccgagtgctgcagaggctgtgcgagcgcggcgcgaagaacgtgctggccttcggcttcgcgctgctggacggggcccgcgggggcccccccgaggccttcaccaccagcgtgcgcagctacctgcccaacacggtgaccgacgcactgcgggggagcggggcgtgggggctgctgttgcgccgcgtgggcgacgacgtgctggttcacctgctggcacgctgcgcgctctttgtgctggtggctcccagctgcgcctaccaggtgtgcgggccgccgctgtaccagctcggcgctgccactcaggcccggcccccgccacacgctagtggaccccgaaggcgtctgggatgcgaacgggcctggaaccatagcgtcagggaggccggggtccccctgggcctgccagccccgggtgcgaggaggcgcgggggcagtgccagccgaagtctgccgttgcccaagaggcccaggcgtggcgctgcccctgagccggagcggacgcccgttgggcaggggtcctgggcccacccgggcaggacgcgtggaccgagtgaccgtggtttctgtgtggtgtcacctgccagacccgccgaagaagccacctctttggagggtgcgctctctggcacgcgccactcccacccatccgtgggccgccagcaccacgcgggccccccatccacatcgcggccaccacgtccctgggacacgccttgtcccccggtgtacgccgagaccaagcacttcctctactcctcaggcgacaaggagcagctgcggccctccttcctactcagctctctgaggcccagcctgactggcgctcggaggctcgtggagaccatctttctgggttccaggccctggatgccagggactccccgcaggttgccccgcctgccccagcgctactggcaaatgcggcccctgtttctggagctgcttgggaaccacgcgcagtgcccctacggggtgctcctcaagacgcactgcccgctgcgagctgcggtcaccccagcagccggtgtctgtgcccgggagaagccccagggctctgtggcggcccccgaggaggaggacacagacccccgtcgcctggtgcagctgctccgccagcacagcagcccctggcaggtgtacggcttcgtgcgggcctgcctgcgccggctggtgcccccaggcctctggggctccaggcacaacgaacgccgcttcctcaggaacaccaagaagttcatctccctggggaagcatgccaagctctcgctgcaggagctgacgtggaagatgagcgtgcggggctgcgcttggctgcgcaggagcccaggggttggctgtgttccggccgcagagcaccgtctgcgtgaggagatcctggccaagttcctgcactggctgatgagtgtgtacgtcgtcgagctgctcaggtctttcttttatgtcacggagaccacgtttcaaaagaacaggctctttttctaccggaagagtgtctggagcaagttgcaaagcattggaatcagacagcacttgaagagggtgcagctgcgggagctgtcggaagcagaggtcaggcagcatcgggaagccaggcccgccctgctgacgtccagactccgcttcatccccaagcctgacgggctgcggccgattgtgaacatggactacgtcgtgggagccagaacgttccgcagagaaaagagggccgagcgtctcacctcgagggtgaaggcactgttcagcgtgctcaactacgagcgggcgcggcgccccggcctcctgggcgcctctgtgctgggcctggacgatatccacagggcctggcgcaccttcgtgctgcgtgtgcgggcccaggacccgccgcctgagctgtactttgtcaaggtggatgtgacgggcgcgtacgacaccatcccccaggacaggctcacggaggtcatcgccagcatcatcaaaccccagaacacgtactgcgtgcgtcggtatgccgtggtccagaaggccgcccatgggcacgtccgcaaggccttcaagagccacgtctctaccttgacagacctccagccgtacatgcgacagttcgtggctcacctgcaggagaccagcccgctgagggatgccgtcgtcatcgagcagagctcctccctgaatgaggccagcagtggcctcttcgacgtcttcctacgcttcatgtgccaccacgccgtgcgcatcaggggcaagtcctacgtccagtgccaggggatcccgcagggctccatcctctccacgctgctctgcagcctgtgctacggcgacatggagaacaagctgtttgcggggattcggcgggacgggctgctcctgcgtttggtggatgatttcttgttggtgacacctcacctcacccacgcgaaaaccttcctcaggaccctggtccgaggtgtccctgagtatggctgcgtggtgaacttgcggaagacagtggtgaacttccctgtagaagacgaggccctgggtggcacggcttttgttcagatgccggcccacggcctattcccctggtgcggcctgctgctggatacccggaccctggaggtgcagagcgactactccagctatgcccggacctccatcagagccagtctcaccttcaaccgcggcttcaaggctgggaggaacatgcgtcgcaaactctttggggtcttgcggctgaagtgtcacagcctgtttctggatttgcaggtgaacagcctccagacggtgtgcaccaacatctacaagatcctcctgctgcaggcgtacaggtttcacgcatgtgtgctgcagctcccatttcatcagcaagtttggaagaaccccacatttttcctgcgcgtcatctctgacacggcctccctctgctactccatcctgaaagccaagaacgcagggatgtcgctgggggccaagggcgccgccggccctctgccctccgaggccgtgcagtggctgtgccaccaagcattcctgctcaagctgactcgacaccgtgtcacctacgtgccactcctggggtcactcaggacagcccagacgcagctgagtcggaagctcccggggacgacgctgactgccctggaggccgcagccaacccggcactgccctcagacttcaagaccatcctggactga

SEQ ID NO: 3 - DNA, 21 bp, Artificial
  Synthetic sequence
aacactgccaacgtccagatg

SEQ ID NO: 4 - DNA, 21 bp, Artificial
  Synthetic sequence
tcgtccagataggcaatgctg

SEQ ID NO: 5 - DNA, 20 bp, Artificial
  Synthetic sequence
agacatcccaccaatcacct

SEQ ID NO: 6 - DNA, 20 bp, Artificial
  Synthetic sequence
agatcacgtcatcgcacaac

SEQ ID NO: 7 - DNA, 19 bp, Artificial
  Synthetic sequence
acgtgatcctcacggcaaa

SEQ ID NO: 8 - DNA, 22 bp, Artificial
  Synthetic sequence
gtgaaaggctcctcaggttctg

SEQ ID NO: 9 - DNA, 20 bp, Artificial
  Synthetic sequence
acccagaagactgtggatgg

SEQ ID NO: 10 - DNA, 20 bp, Artificial
  Synthetic sequence
agaggcagggatgatgttct